(19) United States
(12) Patent Application Publication
(10) Pub. No.: US 2010/0228020 A1
(43) Pub. Date: Sep. 9, 2010

(54) Novel Chlorination Reagent and a Novel Process for Chlorination of Sugars Using Thionyl Chloride

(75) Inventors: Rakesh Ratnam, Karnataka (IN); Sundeep Aurora, Karnataka (IN); Mohammed Mofizuddin, Karnataka (IN)

Correspondence Address: THE NATH LAW GROUP, 112 South West Street, Alexandria, VA, 22314, US

(73) Assignee: V.B Medicare Pvt. Ltd, Karnataka (IN)

(21) Appl. No.: 12/224,324

(22) PCT Filed: Feb. 28, 2007
(86) PCT No.: PCT/IN2007/000076

§ 371 Date: Oct. 20, 2008

(30) Foreign Application Priority Data
Feb. 28, 2006 (IN) 291/MUM/2006

(51) Int. Cl. C07H 13/02; C07H 1/00

(52) U.S. Cl. 536/119; 536/124

(57) ABSTRACT

Preparation of chlorinating reagent or chlorination reaction itself for use in a reaction such as production of high intensity sweetener trichlorogalactosucrose (TGS) from partially protected sucrose, comprising reaction of dimethylformamide (DMF) with thionyl chloride or another sulphur containing inorganic acid chlorides including sulphuryl chloride is faced with a problem of prolific release of gaseous by-products, that at times may lead to violent explosion also. This problem is solved by innovative addition of solid powder inert to the constituents of the chlorination reaction mixture to the reaction, or by adding DMF to acid chloride solution in that order. The invention also leads to use of isolated solid Vilsmeier reagent being used for chlorination in a solvent other than DMF making it possible to avoid altogether problems arising from use of DMF which include irrecoverable loss in alkaline as well as acid conditions, interference in crystallization of TGS and the like.

### TECHNICAL FIELD

The present invention relates to a process of producing a chlorinating reagent using thionyl chloride useful for producing a Vilsmeier-Haack reagent for chlorination of sugars for production of TGS (1′-6′-Dichloro-1′-6′-DIDEOXY-β-Fructofuranasyl-4-chloro-4-deoxy-galacto-pyranoside).

### BACKGROUND OF THE INVENTION

Strategies of prior art methods of production of 4,1′, 6′ trichlorogalactosucrose (TGS) predominantly involve chlorination of sucrose-6-ester by use of Vilsmeier-Haack reagent derived from various chlorinating agents such as phosphorus oxychloride, oxalyl chloride, phosphorus pentachloride etc, and a tertiary amide such as dimethyl formamide (DMF) or dimethyl acetamide to chlorinate Sucrose-6-ester, to form 6 acetyl 4,1′,6′ trichlorogalactosucrose. After the said chlorination reaction, the reaction mass is neutralized to pH 7.0-7.5 using appropriate alkali hydroxides of calcium, sodium, etc. to deesterify/deacetylate the 6 acetyl 4,1′,6′ trichlorogalactosucrose to form 4,1′,6′ trichlorogalactosucrose (TGS). TGS is well known as zero calorie high intensity sweetener.

Vilsmeier-Haack reagent (Vilsmeier hereafter) is derived by reaction of an acid chloride, e.g. phosphorus pentachloride or thionyl chloride, with a tertiary amide, usually dimethylformamide.

The preparation of the Vilsmeier reagent using chlorinating agents such as POCl₃, PCl₅ and a tertiary amide such as N,N-Dimethylformamide (DMF) is well known. However, they generate a large quantity of phosphates as by-products, which are cumbersome to remove and dispose off. Phosgene is a very useful chlorinating agent which can be used for preparation of Vilsmeier reagent by reacting with DMF without getting into problem of generation of phosphate by-products. However, Phosgene is an extremely toxic gas and is an environment safety concern. This leaves possibility of using sulphur containing inorganic acid chlorides, including thionyl chloride and sulphuryl chloride for generation of Vilsmeier reagent by reacting with DMF. However, gaseous by-products in the form of oxides of sulphur are formed when thionyl chloride or sulphuryl chloride react with DMF, and this gaseous byproduct formation also continues when sucrose-6-ester is added to the reaction mixture. This gas evolution is very copious and at any unpredictable time it can take a form of sudden surge leading to a run-away-reaction like situation leading to reactants getting thrown out of reactor. This is a very dangerous element in using thionyl chloride or sulphuryl chloride or other equivalent halides for chlorination reaction. It is necessary to find out safe way of conducting this reaction on a commercial scale for utilizing sulphur containing inorganic acid chlorides for production of TGS. Violent nature of the reactions comprising thionyl chloride as a reactant are very elaborately dealt with by Cardillo (1992) in “Reactivity of thionyl chloride schemes Chim. Ind. (Milan), 1992, 74(12), 879 Lang (ITA)”

This invention describes a novel process for preparation of a novel chlorinating reagent of Vilsmeier type from thionyl chloride or sulphuryl chloride and a new method for chlorinating sugars, including chlorination of sucrose-6-acetate to produce TGS.

An improved method of handling of violent gaseous emissions occurring during a process of the preparation of chlorosugars using Vilsmeier reagent generated by reaction of DMF with thionyl chloride or sulphuryl chloride is being reported.

### THE PRIOR ART

Jenner et al (1982) in U.S. Pat. No. 4,362,869 claimed Vilsmeier reagent of general formula [X CIC.dbd.sup.+NR.sub.2] Cl.sup.—(II) (in which X represents a hydrogen atom or a methyl group and R represents an alkyl group) in an inert solvent, which was obtained by reacting Thionyl chloride (8.5 ml) by adding the same to DMF (8.4 ml) which became hot and the mixture was evaporated under vacuum at 50.degree. to give a syrup. The amount of sulphur dioxide released from such a small scale reaction is very minuscule. Hence, the exothermicity leading to a runaway is not seen. However this reaction if scaled up to a large scale, could lead to the runaway state of reaction.

Mufti et al (1983) in U.S. Pat. No. 4,380,476 have mentioned that “The inorganic acid chloride may be, for example, thionyl chloride, phosphorus oxychloride, or sulphuryl chloride,”. However, thionyl chloride and sulphuryl chloride were not the acid chlorides of their choice, which is indicated by their continuing further in the same statement that “. . . but the acid chloride of choice is phosphorus pentachloride”. They did not report actual steps enabling use of thionyl chloride or sulphuryl chloride for Vilsmeier reagent formation.

Mention has also been made of utility of Thionyl chloride for preparation of Vilsmeier reagent by Rathbone et al in U.S. Pat. No. 4,617,269. They added Thionyl bromide (280 ml) to cooled dimethylformamide (260 ml) with vigorous stirring. The mixture was stirred for 30 minutes at 70.degree.-80.degree. C. and then for a further hour and allowed to cool to ambient temperature. The mixture was filtered and the residue washed with dimethyl formamide (2.times.50 ml) and diethyl ether (100 ml) and dried in a desiccator, to yield 320 g reagent. Here, the reaction was yet a small in size, where the problem of sulphur dioxide evolution could be controlled by vigorous stirring.

O'Brian et al (1988) in U.S. Pat. No. 4,783,526 used triphenylphosphine oxide/thionyl chloride and triphenylphosphine sulfide/thionyl chloride a as a chlorinating reagent for chlorination of carbohydrates and alcohols. Particularly they chlorinated 2,3,6,3′,4′-penta-O-acetylsucrose to obtain 4,1′,6′-trichloro-4,1′,6′-trideoxygalactosucrose pentaacetate for its eventful conversion to 1,6-dichloro-1,6-dideoxy-.beta.-D-fructofuranosyl-4-chloro-4-deoxy-.alpha.-galactopyranoside (TGS). Triphenylphosphine oxide/sulphide are used as recoverable catalysts to reduce side reactions and gaseous by-products are removed by resorting to refluxing for a long period of time such as 2.5 hours to 5 hours and sometimes even higher. Homer et al (1990) in U.S. Pat. No. 4,977,254, chlorinated sugars and partly protected sugar derivatives by reaction of unprotected hydroxyl groups with thionyl chloride to form a persulphite followed by decomposition of the sulphite groups to form chlorosulphites, displacement of the chlorosulphite groups and insertion of chlorine atoms at one or more positions, characterized in that formation and displacement of the chlorosulphite groups and insertion of chlorine atoms is effected by reaction with thionyl chloride in an inert solvent in the presence of a quaternary salt of given general formula. This reaction, however, is not a reaction of preference, because compared to a reaction with a Vilsmeier reagent, e yield from this reaction is much lower, reaction stages are more and handling and isolation of chlorosulphite intermediates are difficult due to their hygroscopic nature.

Walkup et al (1990) in U.S. Pat. No. 4,980,463 in example 13, have described actual method of using thionyl chloride for chlorination in which reaction is provided with a stirrer and a reflux condenser topped with an argon inlet, which suggested that SO2 liberated is removed by bubbling argon gas, the said evolution of SO2 occurring in the steps of reacting sucrose-6-benzoate dissolved in DMF, cooled to −30 degree C., reacting with thionyl chloride added dropwise over a period of 10 minutes, accompanied with rise in temperature to −17 degrees, and heating the reaction mixture thereafter over a period of 15 minutes to 69 degrees. It appears that speed of liberation of SO2 in this case is mainly controlled by lowering the temperature of the reaction mixture to well below sub-zero temperature during entire period of addition of thionyl chloride, the addition of thionyl chloride itself was dropwise, gradual and over an extended period of time, raising the temperature of the reaction mixture thereafter to 69 degrees was also gradual over period of 15 minutes and all these measures were supported further by argon gas bubbling which shall remove the sulphur dioxide gas expeditiously from the reaction mixture without allowing it to build up to a critical concentration to lead to an explosion.

Khan et al (1992) in U.S. Pat. No. 5,136,031 have described a process for the chlorination of sucrose or a derivative thereof, comprising reaction of the sucrose or derivative thereof with thionyl chloride and a nitrogen base at a ratio of about 1 molar equivalent (ME) of thionyl chloride and about 1 ME of base for every ME of free hydroxyl, in a non-reactive moderately polar solvent.

Chlorination of alcohols using thionyl chloride and pyridine has been known since a long time. Gerrard (1940) in J. Chem. Soc. 1939, 998; 218; and 1944, 85 has explained that in a first stage, two alcohol molecules ROH react with thionyl chloride to form a sulphite, and two molecules of hydrogen chloride. Pyridine acts as acid acceptor, which prevents degradation of polysulphite, which reacts with the hydrogen chloride molecules to form pyridine hydrochloride. In a second stage, the sulphite is decomposed by a further reaction with thionyl chloride to provide two molecules of a chlorosulphite. In the third stage, the chlorosulphites react with pyridine hydrochloride to provide two molecules of chloride and two molecules of sulphur dioxide. However, application of this method directly to sugars, which are polyhydroxy compounds leads to complex mixture of products. This problem was aimed to be solved by Khan et al (1992) by their finding that sucrose protected at the 6-position, or sucrose itself, can be reacted with thionyl chloride and a base such as pyridine or an alkyl-substituted pyridine to provide a good yield of the required chlorinated product, provided certain conditions are met. Here too long periods of refluxing perhaps help managing gaseous emissions.

Fairclough, Hough and Richardson in Carbohydrate Research 40 (1975) on pp. 285-298, described chlorination of 4,1′,6′-triol pentaacetate and other partially protected sugars with sulphuryl chloride. The reaction was carried out at subzero temperature of −75 degrees celcius with stirring for four hours and then allowed to attain room temperature. It is clear that gaseous evolution was controlled by using very low temperature accompanied by stirring.

Jenner et al (1982) in U.S. Pat. No. 4,362,869 claimed a process of producing TGS wherein chlorination of 2,3,6,3′,4′-penta-O-acetyl sucrose is done by sulphuryl chloride at a reaction temperature of 20 degrees to 80 degrees C. This chlorinating reagent is used in a mixture of an organic amine base, like pyridine, and a chlorinated hydrocarbon, like chloroform, dichloroethane and the like. The reaction is exothermic and temperature rises to 45 to 55 degrees celcius, is refluxed further for 4 hours and further chlorinated hydrocarbon is added at the end of this step, resulting solution washed successively with hydrochloric acid, water and sodium hydrogen carbonate. The reaction proceeds by formation of chlorosulphate esters followed by their decomposition to form chloro derivatives. Fairclough et al., used a small excess of sulphuryl chloride to ensure complete chlorination and a low temperature, e.g. −75.degree. C. rising eventually to room temperature. Jenner et al found that a greater excess of sulphuryl chloride (e.g. 2-5 ml per 1 g of sucrose pentaacetate as opposed to about 1 ml per 1 ml of pentaacetate) and a much higher reaction temperature (e.g. 20 up to about 55.degree. C. or more) give improved yields, typically to about 75%. However, a disadvantage of this process is that the organic amine, especially pyridine, is prone to get chlorinated by the sulphuryl chloride, leading to the formation of difficult to separate unwanted by-products. In example 8, a successive sue of thionyl chloride as well as sulphuryl chloride in conjunction with trichloroethane and DMF has been described. However, the reaction described involve very small scale, are refluxed for several hours ranging from 5 to 3 hours and addition of reagents is done very slow, all this obviously to control the gaseous emission at high temperatures.

Mufti et al (1983) have also claimed chlorination of monoacylated sucrose derivative with sulphuryl chloride. Mufti et al have disclosed that Vilsmeier reagent is formed when DMF reacts with an inorganic acid chloride, which amongst a list of acid chlorides, is also mentioned to include sulphuryl chloride too. However, whereas actual steps of the reaction are known for other acid chlorides from Mufti et al or prior documents, actual reaction steps are not described for sulphuryl chloride reacting with DMF either by Mufti et al or in later patents. Thus, so far as enabling details about Vilsmeier formation from sulphuryl chloride is concerned, they are described for the first time in this specification.

Mufti et al also mention that sulphuryl chloride itself can be used as chlorinating agent, which reacts initially to form chlorosulphate esters of available hydroxy groups that subsequently or simultaneously decompose with inversion of configuration to provide the corresponding chlorodeoxy derivative. The chlorosulphated intermediates can be isolated in by pouring the reaction mixture into ice-cold sulphuric acid solution and extracting the acid with a solvent. The product obtained may be dechlorosulphated by treatment with a catalytic amount of an iodide such as sodium iodide, preferably in the cold. Mufti et al, however, note that sulphuryl chloride is less selective than the Vilsmeier reagents, which are, hence preferable. This reaction also involved very slow addition of sulphuryl chloride over a period of 1.5 hours maintaining temperature of the reaction to −75 degrees C., pointing to limitations of this reaction and further, pyridine is a noxious solvent, that should be avoided, if possible. The period taken for addition of sulphuryl chloride increases with scale of the reaction, and at commercial scale, it is very clear that slow rate of addition makes the process cumbersome and inefficient.

It is clear from the foregoing that with the requirement that gaseous byproducts release be controlled, with no known method to control it at normal room temperatures, with no known methods by which the time required for addition can be reduced to reasonably short period, with low specificity of sulphuryl chloride/pyridine system to chlorinate partially protected sucrose derivatives, use of sulphuryl chloride as a chlorinating agent has remained impractical although the reaction will not produce cumbersome by-products such as large quantities of inorganic phosphates.

### BRIEF SUMMARY OF THE INVENTION

The present invention describes a novel process based on a new scheme of reaction in which in large production batches involving violent release of gaseous by-products, arising due either to use of thionyl chloride and or another sulphur containing acid chlorides such as sulphuryl chloride as a reactant, is controlled by use of addition of a powder of inert substances, inert to the reactants of a reaction and physically stable under conditions of a reaction when thionyl halides, particularly thionyl chloride, and sulphuryl chloride are used in a reaction scheme for chlorination of compounds having organic compounds having hydroxy group in general and partially protected sucrose and their derivatives in particular. Such a powder includes, without being limited to, an adsorbent or an inert matter and the like. The said adsorbent includes, without being limited to, Activated Carbon, Zeolite and the like. The said inert matter may include, diatomaceous earth, silica, calcium aluminium silicates and the like.

In one embodiment of the invention, N,N-dimethyl formiminium chloride chlorosulphite or N,N-dimethyl formiminium chloride chlorosulphate adducts formed respectively from thionyl chloride and sulphuryl chloride are formed in reaction with DMF in presence of an adsorbent. In a preferred embodiment, chlorosulphite adduct formed from thionyl chloride is used as in-situ generated product itself because it does not solidify at room temperature. The corresponding adduct, chlorosulphate formed from sulphuryl chloride with DMF solidifies easily at room temperature and can be separated and used later and hence can be used as a separated reagent or as formed in in-situ form itself without separating out.

In another embodiment of the invention, release of the gaseous byproducts can also be controlled by reversing the order of addition i.e. adding DMF to either thionyl chloride or sulphuryl chloride rather than vice-versa. Gases are released in controlled manner and Vilsmeier reagent is produced in a safe way. In this embodiment the reaction is spontaneous and the sulphur dioxide evolved is not accumulated in the reaction mass. This is evolved instantaneously and nitrogen sparging helps in removing the gases evolved without any help of adsorbent. Hence nitrogen sparging itself is enough in the case of reverse addition for the removal of sulphur dioxide. This reaction specifically doesn't require addition of adsorbents and removal of sulphur dioxide takes place peacefully.

In yet another embodiment, Vilsmeier regent is allowed to be formed completely by using one or both of above improvements to avoid surge of gaseous byproducts and the reagent formed in this way is separated or used in-situ for chlorination, so that no gaseous byproducts evolve during chlorination process.

In yet another embodiment of this invention, addition of reactants including thionyl chloride, sulphuryl chloride and partially protected sugar derivatives to a reaction mixture containing DMF can be done at much faster rate than was possible in prior art processes without addition of adsorbents or without reversing the addition of DMF as referred above and at a temperature exceeding zero degrees C.

In yet another embodiment of this invention, the Vilsmeier formation is very effective and the reaction goes to completion faster if thionyl chloride or sulphuryl chloride is added to DMF at temperatures above 30° C. The maintenance of reaction mass at that temperature after addition also is related to the complete Vilsmeier formation.

In yet another embodiment of this invention, the chlorosulphite reagent and chlorosulphate reagent formed respectively from reaction with DMF of thionyl chloride and sulphuryl chloride are capable of performing chlorination reaction in other solvents than DMF, such as in DMSO (Dimethylsulphoxide), pyridine perchloroethylene and the like. The advantage is that these solvents do not participate in the reaction, hence, do not convert to other forms and can be recovered easily after the reaction is completed making solvent recovery possible without any loss rendering the process highly efficient and economical. The other advantage is some of these solvents are highly stable and do not undergo decomposition at higher temperature or at higher pH unlike DMF. Further, DMF is known to be difficult to recover and separate from the product totally; this problem gets automatically eliminated with no need to use DMF. Further, avoiding use of DMF has further advantages because at elevated temperatures during chlorination and quenching considerable quantity of DMF degrades which is an irrecoverable loss.

The TGS formed in the process can be purified and isolated using one or more of a method including, but not limited to, filtration through a filter press, ultrafiltration, reverse osmosis, molecular filtration, column chromatography including hydrophobic and affinity chromatography, solvent extraction, crystallization, precipitation in an organic solvent to form an amorphous powder, or a microcrystalline powder or a mixture of several forms of powder and the like.

### BRIEF DESCRIPTION OF THE DRAWINGS

FIG. 1A shows structural formula of N,N-dimethyl formiminium chloride chlorosulphite reagent.

FIG. 1B shows structural formula of N,N-dimethyl formiminium chloride chlorosulphite reagent.

FIG. 1C shows structural formula of Vilsmeier reagent formed from thionyl chloride and sulphuryl chloride.

### DETAILED DESCRIPTION OF THE INVENTION

A new scheme of chlorination of partially protected sucrose and their derivatives is found out in this invention.

It was seen in the scheme of the reaction invented here, that thionyl chloride and sulphuryl chloride can be used interchangeably to reach a same effect or to achieve a same objective.

Similarly, although present preference is to use partially protected sucrose, particularly sucrose-6-ester as raw material for a further process of production of TGS, this invention is also applicable to and includes within the scope of its claims, any process that envisages starting a reaction with a sugar as raw material comprising use of a sulphur containing acid chloride or their derivatives for chlorination.

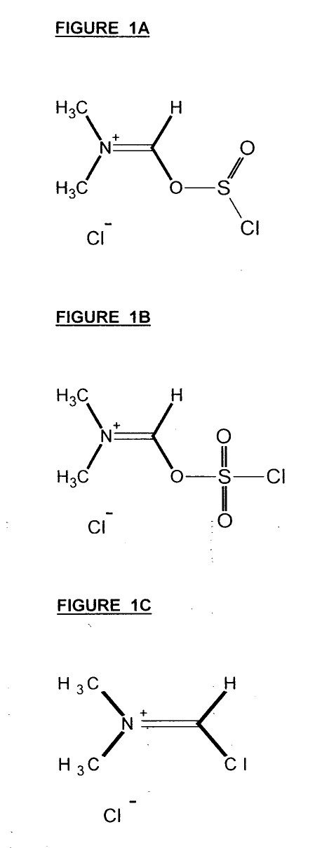

When Thionyl chloride reacts with a tertiary amide such as DMF, it leads to formation of a reagent which can be described as N,N-dimethylformiminium chloride chlorosulphite (hereinafter designated as “chlorosulphite reagent” which is shown in FIG. 1A.

Similarly, it was found that when sulphuryl chloride reacts with dimethyl formamide, essentially similar reagents N,N-dimethyl formiminium chloride chlorosulphate (hereinafter designated as “chlorosulphate reagent” or simply as “chlorosulphate”) is formed, the structure of which is shown in FIG. 1B.

The chlorosulphite reagent and chlorosulphate reagent themselves have similar properties and offer same strategies for their utility in a process for chlorination of partially protected sucrose derivatives. Both are capable of carrying out chlorination, The chlorosulphite reagent formed from thionyl chloride is stable only at very low temperatures, can not be isolated as solid at room temperature and has to be used as in-situ generated reagent itself. At higher temperatures required for chlorination reaction, It is unstable and releases sulphur dioxide. The reagent formed from sulphuryl chloride is stable at room temperature, can be isolated as a solid and can be used as separated reagent for chlorinating sugar derivatives. This reagent also releases gaseous byproduct, sulphur trioxide, when heated during chlorination reaction, which can be removed from the said reagent or from the reaction mixture containing the said reagent by suitable methods such as suction through vacuum, heating to elevated temperature, etc and then the subsequent Vilsmeier formed after liberation of gaseous byproducts is taken for chlorination.

The chlorosulphite reagent and chlorosulphate reagent formed respectively from reaction with DMF of thionyl chloride and sulphuryl chloride are capable of performing chlorination reaction in solvents other than DMF, such as in DMSO (Dimethylsulphoxide), pyridine, tetrachloroethane, perchloroethylene and the like. The advantage is that these solvents do not participate in the reaction, hence, do not convert to other forms and can be recovered easily after the reaction is completed making solvent recovery possible without any loss rendering the process highly efficient and economical. . . . Further, DMF is known to be difficult to recover and separate from the product totally; this problem gets automatically eliminated with no need to use DMF. Further, avoiding use of DMF has further advantages because at elevated temperatures during chlorination and quenching considerable quantity of DMF degrades which is an irrecoverable loss, which is avoided when use of DMF is avoided altogether Still further, DMF is a high boiling solvent and its recovery is a bottleneck. Still further, DMF is a solvent which degrades at higher temperature and at extreme pH ranges. The reaction mass during chlorination is heated to elevated temperatures and the pH of the reaction mass is highly acidic during chlorination. Further during the quenching of the reaction mass, the mass is exposed to alkaline pH also. During all these operations, the DMF is exposed to harsh conditions and degrades up to various levels and is lost forever. This will not be the case when we use other solvents, which are more stable and also that do not participate in the reaction.

Vilsmeier reagent formed from thionyl chloride and sulphuryl chloride in this way is same as Vilsmeier reagent formed from other acid chlorides such as phosphorus pentoxide, phosphorus oxychloride, phosgene etc. Whatever gaseous evolution occurs, it is limited to formation of this Vilsmeier regent, and once the gaseous by-products have been removed from the reaction mixture or the Vilsmeier reagent is separated and used, later reaction is same as chlorination by Vilsmeier reagent formed from any other chlorinating agent. Here too, in the reaction with the isolated Vilsmeier reaction, DMF can be avoided and other suitable solvents such as tetrachloroethane, perchloroethylene, toluene, etc. can be used as the reaction medium

Thus, if the problem of sudden/unpredictable and violent surge of gases during the reaction is reliably prevented, use of Thionyl Chloride or Sulphuryl chloride as acid chlorides for Vilsmeier reagent preparation and further chlorination of partly protected sucrose derivatives shall be a better choice when compared to other usually used acid chlorides for Vilsmeier reagent preparation, such as Phosgene, which is highly toxic and hazardous and Phosphorus oxychloride or Phosphorus pentachloride which generate difficult to manage phosphate by-products.

The putative structure of the Vilsmeier type reagent formed after liberation of SO₂ in case of chlorosulphite reagent, or SO₃ in case of chlorosulphate reagent, is given in FIG. 1C.

Liberation of Sulphur dioxide from chlorosulphite reagent or sulphur trioxide from chlorosulphate reagent is highly critical if it occurs during the heating cycle of chlorination, since it is extremely violent and the reaction mixture tends to bump out of the reactor. The rate of evolution of the gas from the reaction mass is very high and contributes to higher pressures and eventually becomes a runaway type of reaction.

This invention discloses a novel way of controlling the rate of evolution of the gas from the reaction by addition of a suitable adsorbent or tiny solid particles of material that is inert (hereafter referred to as “inert particles” or “inert substances”) to the reaction mixture or remains unaffected adversely under reaction conditions. It is thought that this adsorbent or inert particles trap the gas as soon as it is released from chlorosulphite reagent or chlorosulphate reagent, perhaps on account of sheer physical adsorption due to physical attractive forces in matter which is totally inert or in case of adsorbents, on account of additional attractive forces, and avoids its sudden violent release. The adsorbent or inert particles act as a via media or as an interphase for the release of gas from the chlorosulphite reagent or chlorosulphate reagent to itself and subsequently by desorption to the reaction mixture and then to the scrubber, thus controlling the rate of evolution of sulphur dioxide/sulphur trioxide. Possible mechanism of action is that the adsorption and desorption takes place simultaneously and results in controlling the rate of evolution of gas. The inert powder comprising adsorbent or inert substances may preferably of a size ranging from 5 micron/ to 350 micron/, most preferably from 50 micron/mm to 100 micron/mm.

An adsorbent used usually includes, without being limited to, Activated Charcoal, Zeolites and the like. Inert substances, inert towards the reactants of this invention also includes, without being limited to, diatomaceous earth, silica, calcium aluminium silicates, and the like. The adsorbent and/or the inert particulate matter added plays a role only with sulphur dioxide/sulphur trioxide adsorption and is relatively inert to the chlorination reaction.

Of course, addition of adsorbents/inert particles serve the purpose even when added in reactions which are run at lower temperature.

After the chlorination reaction, the reaction mass along with the adsorbent is neutralized and then filtered. The adsorbent can be separated in the filter cake and regenerated. Because gaseous evolution is controlled in this way, it becomes possible to use thionyl chloride based and sulphuryl chloride based chlorination reactions at much higher temperatures than were possible with prior art, reagent addition period is comparatively as well as reasonably less; there is no need of measures such as refluxing for a long time, vigorous stirring and the like; Vilsmeier reagent formation also goes to completion faster because reactions are conducted at around room temperature.

The examples described below serve as illustration on how to practice the invention claimed in this specification and do not limit the scope of actual techniques used or scope of or range of reaction conditions or process conditions claimed. Several other adaptations of the embodiments will be easily anticipated by those skilled in this art and they are also included within the scope of this work. Mention of a singular also includes pleural of the same. Thus a mention of “a solvent” also includes more than one solvent. Equivalent alternatives of a reactant or a reaction condition are also included within the scope of claims of this specification. Thus, mention of “chloride” also includes other halides, such as a bromide, if they can perform same function, if used as an alternative chemical. Similarly, a mention of “an ester of sucrose” includes in it monoester as well as pentaesters and their derivatives. In general, any modification or an equivalent obvious to a person skilled in the art is included within the scope of this specification and its claims.

This reagent is formed when sequentially (a) DMF (7.0-12 moles) is taken in a flask at temperature between −10 to 35 degrees celcius, (b) adsorbent or inert particles or both are added in a quantity which is 5 to 15% of weight of thionyl chloride input, (c) thionyl chloride (about 3.5 to 5 moles or above) is added to the flask slowly over a period of time at room temperature maintaining temperature of the reactants to preferably below 30° C.

Then the 6-acyl sucrose is added to the mass below 5° C. temperature, controlling the temperature to below 5° C. and then the reaction mass is heated to 35° C., maintained for 60 minutes. Then the reaction mass is heated to 85° C. and maintained for 60 minutes and then again temperature raised to 100° C. and maintained for 6 hours. Then finally the temperature is raised to 115° C. and maintained for 1.5 hrs and then cooled to 60° C.

The reaction mass is then neutralized using 7% Ammonia solution in water up to pH 7.0. The neutralized mass is then filtered and the suspended solids are separated. The adsorbent is also separated at this stage and is heated to elevated temperatures for activation and re-use. Most of the gaseous oxides of sulphur liberated during the reaction are let out of the reaction mixture to the scrubber and the rest forms the inorganic salts with ammonia. The amount of inorganic compounds formed after chlorination during neutralization is far less than any of the use of chlorinating reagents used such as Phosphorus pentachloride or Phosphorus oxychloride.

In another embodiment, the tertiary amide such as DMF is added in equimolar quantity to the acid chloride such as thionyl chloride or sulphuryl chloride at a temperature between 35-50° C. The DMF is added slowly and it reacts instantly with the acid chloride forming a chlorosulphite or chlorosulphate reagent salt. This in turn is instantly converted to the Vilsmeier reagent with the release of sulphur dioxide or sulphur trioxide respectively. Here the process of release of sulphur dioxide or sulphur trioxide gases is controlled since it is controlled by the addition of DMF to the reaction mass. In this embodiment, the requirement of an adsorbent is avoided and the Vilsmeier is allowed to be formed slowly at the said temperature with the addition of DMF. After the said Vilsmeier is formed, the reaction mass is cooled to −5° to 5° C. and the sucrose-6-acetate solution in DMF is added. Then the reaction mass is heated to ambient and heated to elevated temperatures to facilitate chlorination for the preparation of TGS.

In all the above embodiments the nitrogen sparging is carried out to remove gases liberated from the reaction mass in an efficient manner The nitrogen sparging in the reaction plays an important role in scavenging the gases from the reaction mixture. It also prevents moisture entertainment into the reaction mass. However it doesn't independently control the rate of emission of gases from the reaction mass.

An adsorbent or an inert substance, however, have adsorption capacity/physical affinity towards the sulphur dioxide or sulphur trioxide evolved during the chlorination reaction and hence control the rate of evolution of these gases.

Vilsmeier formation at higher temperature and maintenance of temperature at about 25 to 35 degrees is an embodiment of this invention which accomplishes complete reaction of thionyl chloride with DMF unlike other reactions where some thionyl chloride remains unreacted.

It may be mentioned here that 6-protected sucrose that can be chlorinated by this invention can also be selected, in addition to sucrose-6-ester, also from 6-ether and a 6,4-diester. The said 6-protected sucrose can also be selected, in addition to 6-acetate, also from 6-benzoate and raffinose. The process for the preparation of TGS may also comprise chlorination of sucrose-6-esters by using Vilsmeier-type reagent by a process to form a sucralose-6-ester, esterification and de-esterification of the pentaester to form TGS.

Thionyl chloride and sulphuryl chloride are easier to handle compared to other chlorinating agents at the industrial scale and provides way for a more economically viable process.

### Example 1

### Chlorination Using Thionyl Chloride

460 L of DMF was taken in a Glass Lined Reactor (GLR) followed by addition of 16 kg of Charcoal. The mixture was stirred and 344 kg of thionyl chloride was added to the reactor through the dosing tank over a period of 60 minutes. A nitrogen sparger line was fitted to the reactor and sparging of nitrogen was continued throughout the reaction.

The temperature was maintained between 35-40° C. After the addition of thionyl chloride the mass was stirred for 60 minutes and then cooled to 0° C. 80 kg of 86% sucrose-6-acetate solution in DMF was added over 10-15 hours to the reaction mass and the temperature was controlled below 5° C.

The mass was then allowed to attain room temperature of 30° C. and then stirred for 60 minutes. Then the mass was heated to 85° C. slowly over a period of 3 hours and maintained at 85° C. for 60 minutes and then heated to 100° C. and maintained for 6 hours. Then the mass was further heated to 114° C. and maintained for 1.5 hrs and cooled to 60° C.

The mass was then neutralized with 7% ammonia solution to pH 7.0. The neutralized mass was analyzed for 6-acetyl TGS and was found to be 65% of sucrose input.

The neutralized mass was then filtered in a filter press to remove the suspended solids. The filtrate obtained was then passed through an affinity chromatographic column containing the Thermax ADS 600 resin. The TGS-6-acetate was adsorbed on to the resin and the DMF along with inorganic salts passed out of the resin.

The adsorbed TGS-6-acetate was desorbed using 10% ammonia solution in methanol. The in-situ deacylation of TGS-6-acetate to TGS also takes place during desorption.

The TGS solution in ammonia methanol is then neutralized by addition of dilute HCl. The neutralized solution was then distilled to remove methanol. The syrup obtained was treated with ethyl acetate and methanol and crystallized.

The overall yield obtained from sucrose-6-acetate input was found to be 35%.

### Example 2

### Chlorination Using Sulphuryl Chloride

### Chlorination of Sucrose-6-benzoate Using Sulphuryl Chloride

2100 ml of DMF was taken in a round bottom reaction flask and 80 g of Charcoal was added to it and was stirred. The nitrogen sparging was started in the flask. The temperature was held at 25° C. and 1100 ml of sulphuryl chloride was added dropwise through an addition funnel. After the completion of addition, the reaction mass was held at 25° C. under stirring for 60 minutes. Then the mass was cooled to 0° C. and 1000 ml of sucrose-6-benzoate (340 g) solution in DMF. The temperature was controlled below 5° C. After the completion of addition of sucrose-6-benzoate, the reaction mass was stirred for 3.5 hours at 25-30° C. Then the reaction mass was heated to 85° C. and maintained for 1 hour, again heated to 100° C. and maintained for 6 hours and further heated to 115° C. and maintained for 2.5 hours.

The reaction mass was then cooled to 60° C. and was neutralized with 7% ammonia solution. The neutralized mass containing 6-benzoyl TGS was filtered to remove the suspended solids along with the charcoal. During the reaction, the evolution of sulphur trioxide was very peaceful and no sudden surge of gases was seen. The overall yield of 6-benzoyl TGS over sucrose-6-benzoate input was analyzed to be 66%.

### Example 3

### Isolation of N,N-dimethylformiminium Chloride Chlorosulphate Prepared from Sulphuryl Chloride

1150 ml of Methylene dichloride was taken in reaction flask. 982 g of sulphuryl chloride was added dropwise over a period of 3 hours at temperature below −20° C. A nitrogen sparger line was fitted to the flask and sparging of nitrogen was continued throughout the reaction. The reaction mixture was stirred continuously. Then 520 g of DMF was added slowly to the reaction mixture with vigorous stirring and temperature maintained below −20° C.

Then the temperature was slowly raised to 0° C. The adduct N,N-dimethyl formiminium chloride chlorosulphate was formed precipitated out of the solution. The mixture was vigorously stirred at this temperature and was filtered out in cold condition and was stored under liquid nitrogen till further use.

### Example 4

### Chlorination of Sucrose-6-benzoate Using the Isolated N,N-dimethylformiminium Chloride Chlorosulphate

The isolated N,N-dimethylformiminium chloride chlorosulphate adduct 902.8 g was taken in a reaction flask and 1200 ml of Dimethyl sulphoxide was added. 40 g of activated zeolite was added to the mixture and was kept stirring. A nitrogen sparger line was fitted to the flask and sparging of nitrogen was continued throughout the reaction. and the temperature was maintained between 0 to 5° C. 176 g of sucrose-6-benzoate was added to the reaction mixture and was stirred for 60 minutes. The temperature was controlled during the addition of sucrose-6-benzoate below 5° C.

The reaction mass (or alternatively designated hereafter as “mass”) was then allowed to attain room temperature and maintained for 60 minutes. Then the mass was heated to 85° C. slowly over a period of 3 hours and maintained at 85° C. for 60 minutes and then heated to 100° C. and maintained for 6 hours. Then the mass was further heated to 114° C. and maintained for 1.5 hrs and cooled to 60° C.

The mass was then neutralized with 7% ammonia solution to pH 7.0. The neutralized mass obtained was 7 L, which was analyzed for TGS-6-benzoate and was found to be 109.9 g (64.6% conversion of sucrose-6-benzoate input).

### Example 5

### Reverse Addition of DMF to Acid Chloride

520 ml of thionyl chloride was taken in a 3 necked round bottom flask. A nitrogen sparger line was fitted to the flask. The temperature was maintained at 35-40° with stirring. Then 550 ml of DMF was added dropwise to the mass and the temperature was controlled below 50° C. by active cooling whenever required over 3-hours. The nitrogen was kept sparging throughout the DMF addition

When the reaction was taking place, continuous evolution of sulphur dioxide fumes from the reaction was observed. This was tested by exposing the fumes to paper dipped in potassium dichromate solution. The change of color from yellow to green indicates the evolution of sulphur dioxide.

After the completion of the addition of DMF, the reaction mass was held at 45-50° C. for 3 hours to facilitate complete removal of sulphur dioxide. This was confirmed by treating the reaction mass with potassium dichromate solution. If the green colour was not formed in the solution, this indicates the complete removal of sulphur dioxide from the reaction mass.

Then the reaction mass was cooled to 0-5° C. and 900 ml containing 22% sucrose-6-acetate solution in DMF was added dropwise under stirring. The reaction mass was then allowed to attain room temperature and maintained for 60 minutes. Then the mass was heated to 85° C. slowly over a period of 3 hours and maintained at 85° C. for 60 minutes and then heated to 100° C. and maintained for 6 hours. Then the mass was further heated to 114° C. and maintained for 1.5 hrs and cooled to 60° C.

The mass was then neutralized with 7% ammonia solution to pH 7.0. The neutralized mass obtained was 7 L, which was analyzed for TGS-6-acetate and was found to be 90 g.

### Example 6

### Isolation of N,N-dimethyl Formiminium Chloride Salt from Thionyl Chloride

460 L of DMF was taken in a Glass Lined Reactor followed by addition of 12 kg of zeolite adsorbent. The mixture was stirred and 344 kg of thionyl chloride was added to the reactor through the dosing tank over a period of 60 minutes. A nitrogen sparger line was fitted to the reactor and sparging of nitrogen was continued throughout the reaction. The temperature was maintained between 35-40° C. After the addition of thionyl chloride the mass was stirred for 5 hours and then slowly the temperature was increased up to 70° C. over a period of 5 hours and the complete elimination of sulphur dioxide gas was analyzed. This was tested by exposing the fumes to paper dipped in potassium dichromate solution. The change of color from yellow to green indicates the evolution of sulphur dioxide.

Then the reaction mass was cooled to 15° C. and the mass started precipitating a solid. The precipitation was allowed to complete in 3 hours. This solid along with adsorbent was filtered off using a closed filtration system under nitrogen. The solids filtered was carefully transferred back to the washed GLR and the quantity weighed was 518.5 kg. This solid N,N-dimethyl formiminium chloride salt was used for chlorination of sucrose-6-acetate.

### Chlorination of Sucrose-6-acetate Using Isolated N,N-dimethyl Formiminium Chloride Salt

The said isolated solid N,N-dimethyl formiminium chloride salt was taken in the GLR and was cooled to 0° C. 500 L of DMF was added in the reaction mass and was kept stirring. A nitrogen sparger line was fitted to the reactor and sparging of nitrogen was continued throughout the reaction. 80 kg of 82% sucrose-6-acetate solution in DMF was added over 10-15 hours to the reaction mass and the temperature was controlled below 5° C.

The mass was then allowed to attain room temperature of 30° C. and then stirred for 60 minutes. Then the mass was heated to 85° C. slowly over a period of 3 hours and maintained at 85° C. for 60 minutes and then heated to 100° C. and maintained for 6 hours. Then the mass was further heated to 114° C. and maintained for 1.5 hrs and cooled to 60° C.

The mass was then neutralized with 7% ammonia solution to pH 7.0. The neutralized mass was analyzed for 6-acetyl TGS and was found to be 54% of sucrose input. The DMF loss in the reaction was found to be 20% of the input

### Example 7

### Isolation of N,N-dimethyl Formiminium Chloride Salt from Sulphuryl Chloride

560 L of DMF was taken in a Glass Lined Reactor followed by addition of 12 kg of charcoal adsorbent. The mixture was stirred and 208 kg of sulphuryl chloride was added to the reactor through the dosing tank over a period of 60 minutes. A nitrogen sparger line was fitted to the reactor and sparging of nitrogen was continued throughout the reaction. The temperature was maintained between 35-40° C. After the addition of sulphuryl chloride the mass, was stirred for 5 hours and then slowly the temperature was increased up to 85° C. over a period of 9 hours and the complete elimination of sulphur trioxide gas was analyzed. This was tested by exposing the fumes to paper dipped in potassium dichromate solution. The change of color from yellow to green indicates the evolution of sulphur trioxide.

Then the reaction mass was cooled to 15° C. and the mass started precipitating a solid. The precipitation was allowed to complete in 3 hours. This solid along with adsorbent was filtered off using a closed filtration system under nitrogen. The solids filtered was carefully transferred back to the washed GLR and the quantity weighed was 100 kg.

This solid N,N-dimethyl formiminium chloride salt was taken in the GLR and was cooled to 0° C. 500 L of DMF was added in the reaction mass and was kept stirring. 65 kg of 82% sucrose-6-acetate solution in DMF was added over 10-15 hours to the reaction mass and the temperature was controlled below 5° C.

The mass was then allowed to attain room temperature of 30° C. and then stirred for 60 minutes. Then the mass was heated to 85° C. slowly over a period of 3 hours and maintained at 85° C. for 60 minutes and then heated to 100° C. and maintained for 6 hours. Then the mass was further heated to 114° C. and maintained for 1.5 hrs and cooled to 60° C.

The mass was then neutralized with 7% ammonia solution to pH 7.0. The neutralized mass was analyzed for 6-acetyl TGS and was found to be 60% of sucrose input.

### Example 8

Chlorination of Sucrose-6-acetate Using Isolated N,N-dimethyl Formiminium Chloride Salt in Perchloroethylene

The isolated solid from Example 5 was taken in the GLR. 500 L of perchloroethylene was added in the reaction mass, cooled to 0° C. and was kept stirring. A nitrogen sparger line was fitted to the reactor and sparging of nitrogen was continued throughout the reaction. 80 kg of 82% sucrose-6-acetate solution in perchloroethylne was added over 10-15 hours to the reaction mass and the temperature was controlled below 5° C. The mass was then allowed to attain room temperature of 30° C. and then stirred for 60 minutes. Then the mass was heated to 85° C. slowly over a period of 3 hours and maintained at 85° C. for 60 minutes and then heated to 100° C. and maintained for 6 hours. Then the mass was further heated to 114° C. and maintained for 1.5 hrs and cooled to 60° C.

The mass was then neutralized with 7% ammonia solution to pH 7.0. The neutralized mass was analyzed for 6-acetyl TGS and was found to be 54% of sucrose input. The Perchloroethylene in the neutralized mass was analyzed and the loss was found to be 3.5% of the input.

### Example 9

Chlorination of Sucrose-6-benzoate Using Thionyl Chloride in the Presence of Diatomaceous Earth

1150 ml of DMF was taken in a round bottom reaction flask and 40 g of diatomaceous earth was added to it and was stirred. The nitrogen sparging was started in the flask. The temperature was held at 25° C. and 520 ml of thionyl chloride was added dropwise through an addition funnel. The temperature was controlled below 30° C. After the completion of addition of thionyl chloride, the reaction mass was stirred for 60 minutes at 25-30° C. and was cooled to 0° C. 900 ml of sucrose-6-benzoate (170 g) solution in DMF was added with stirring. The temperature was controlled below 5° C. After the addition of sucrose-6-benzoate solution, the reaction mass was allowed to attain room temperature and stirred for 3 hours.

Then the reaction mass was heated to 85° C. and maintained for 1 hour, again heated to 100° C. and maintained for 6 hours and further heated to 115° C. and maintained for 2.5 hours.

The reaction mass was then cooled to 60° C. and was neutralized with 7% ammonia solution. The neutralized mass containing 6-benzoyl TGS was filtered to remove the suspended solids along with the diatomaceous earth. During the reaction, the evolution of sulphur dioxide was very peaceful and no sudden surge of gases was seen. The overall yield of 6-benzoyl TGS over sucrose-6-benzoate input was analyzed to be 68%.

### Example 10

### Chlorination of Sucrose-6-benzoate Using Thionyl Chloride Changing the Sequence of Addition i.e. Adding Chlorinating Agent to Sucrose-6-ester

2100 ml of DMF was taken in a round bottom reaction flask and 80 g of Charcoal was added to it and was stirred. 1000 ml of sucrose-6-benzoate (340 g) solution in DMF was added with stirring. The nitrogen sparging was started in the flask. The temperature was held at 25° C. and 1040 ml of thionyl chloride was added dropwise through an addition funnel. The temperature was controlled below 30° C. After the completion of addition of thionyl chloride, the reaction mass was stirred for 3.5 hours at 25-30° C. Then the reaction mass was heated to 85° C. and maintained for 1 hour, again heated to 100° C. and maintained for 6 hours and further heated to 115° C. and maintained for 2.5 hours.

The reaction mass was then cooled to 60° C. and was neutralized with 7% ammonia solution. The neutralized mass containing 6-benzoyl TGS was filtered to remove the suspended solids along with the charcoal. During the reaction, the evolution of sulphur dioxide was very peaceful and no sudden surge of gases was seen. The overall yield of 6-benzoyl TGS over sucrose-6-benzoate input was analyzed to be 48%.

### Example 11

### Chlorination of Sucrose-6-acetate Using Thionyl Chloride in the Presence of Diatomaceous Earth Without Nitrogen Sparging

1000 ml of DMF was taken in a round bottom reaction flask and 40 g of diatomaceous earth was added to it and was stirred. The temperature was held at 25° C. and 480 ml of thionyl chloride was added dropwise through an addition funnel. The temperature was controlled below 30° C. After the completion of addition of thionyl chloride, the reaction mass was stirred for 60 minutes at 25-30° C. and was cooled to 0° C. 1050 ml of sucrose-6-acetate (180 g) solution in DMF was added with stirring. The temperature was controlled below 5° C. After the addition of sucrose-6-acetate solution, the reaction mass was allowed to attain room temperature and stirred for 3 hours. Then the reaction mass was heated to 85° C. and maintained for 1 hour, again heated to 100° C. and maintained for 6 hours and further heated to 115° C. and maintained for 2.5 hours.

The reaction mass was then cooled to 60° C. and was neutralized with 7% ammonia solution. The neutralized mass containing 6-acetyl TGS was filtered to remove the suspended solids along with the diatomaceous earth. During the reaction, the evolution of sulphur dioxide was peaceful and no sudden surge of gases was seen. The overall yield of 6-acetyl TGS over sucrose-6-acetate input was analyzed to be 55%.

1. A process of controlling prolific emission of a gaseous by-product in a reaction by addition to the said reaction mixture of a powder of substances inert towards reactants and stable under conditions of reaction, the said powder being capable of controlling the said prolific emission of gases to a safe release, the said addition being made after addition of first solution of reactants to a reactor in a quantity sufficient to control the said prolific emission without allowing sudden uncontrollable surge of gases, the said reaction comprising one or more of a reaction including (A) reaction of a sulphur containing inorganic acid chloride with a tertiary amide resulting in a first reaction mixture containing an adduct formed between the said acid chloride and the tertiary amide, or (B) a chlorination reaction by reacting the said adduct either after its isolation from the said first reaction mixture, the said chlorination reaction being carried in a medium suitable for progress of a chlorination reaction with a sugar or a partially protected sugar, or (C) a chlorination reaction comprising reacting the adduct existing as an in-situ reagent in the said first reaction mixture with a sugar or a partially protected sugar to prepare a chlorinated sugar derivative, or (D) heating the said first reaction mixture to form a second reaction mixture comprising decomposition of the said adduct in-situ to form a Vilsmeier reagent of general formula [XCIC=N.sup.+R.sub.2]Cl.sup.—wherein R represents an alkyl group, typically a methyl or ethyl group, and X represents a hydrogen atom or a methyl group, and optionally isolating the solid Vilsmeier reagent formed or (E) a chlorination reaction by reacting the said second reaction mixture with in-situ formed Vilsmeier reagent with a partially protected sugar to prepare a chlorinated sugar derivative, or (F) a chlorination reaction by reacting a sugar or partially protected sugar dissolved in DMF with a sulphur containing inorganic acid chloride followed optionally by isolating the said chlorinated sugar derivative or deacylating the said chlorinated sugar derivative to form Trichlorogalactosucrose (TGS).

2. A process of claim 1, wherein:
a. the said gaseous by product is an oxide of sulphur, including sulphur dioxide or sulphur trioxide,
b. the said powder including (i) a matter capable of having a physical adsorption ability or a loose chemical affinity without leading to a chemical change or both with respect to the an emitting gas, (ii) being stable under conditions of the said one or more of a said reaction, and (iii) being inert towards constituents of the reactants of a said reaction; comprising one or more of an adsorbent or an inert matter including charcoal, diatomaceous earth, silica, calcium aluminium silicates, and the like,
c. the said quantity of powder added is about 5 to 15%, preferably about 5 to 5% by weight of the weight of the said acid chloride,
d. the said sulphur containing inorganic acid chloride comprises one or more of a thionyl chloride, sulphuryl chloride and the like,
e. the said tertiary amide comprising one or more of a dimethylformamide (DMF), Dimethyl acetamide, and the like,
f. the said adduct comprising N,N-dimethylformiminium chloride chlorosulphite (chlorosulphite reagent) when the acid chloride used in its generation is thionyl chloride or N,N-dimethylformiminium chloride chlorosulphate (chlorosulphate reagent) when the acid chloride used in its generation is sulphuryl chloride,
g. the said medium suitable for progress of a chlorination reaction being an organic solvent comprising one or more of a DMF, dimethyl sulfoxide, perchloroethylene, Toluene, Pyridine, xylene and the like,
h. the said sugar comprises raffinose,
i. the said partially protected sugar comprises a pentaester of sucrose or a 6-protected sucrose, the 6-protected sucrose further comprising a sucrose-6-ester or a sucrose-6-ether or a sucrose-6-diester, further including sucrose-6-acetate, sucrose-6-benzoate, sucrose-6,4′ diester, 2,3,6,3′,4′-pentaester, a sucrose-6-glutarate, sucrose-6-propionate, sucrose-6-laurate, sucrose-6-phthalate, a sucrose-6-methyl ether, sucrose-6-ethyl ether and the like,
j. the said process of purification and isolation includes one or more of a process comprising solvent extraction, column chromatography, reverse osmosis, crystallization and the like.

3. A process of claim 2 of production of TGS comprising use of either thionyl chloride or sulphuryl chloride as a preferred sulphur containing acid chloride to generate a chlorinating reagent, the said process further comprising following sequential steps: (A) taking DMF as a preferred tertiary amide in a reactor, optionally sparging nitrogen as a preferred inert gas through the reaction mixture, (B) adding to it charcoal powder as a preferred powder for controlling gaseous emissions, (C) adding to the reaction mixture, the said preferred sulphur containing acid chloride slowly over a period of time maintaining temperature preferably between about 35° to 40° C., (D) maintaining the reaction mass preferably stirred for a preferred period of about 60 minutes for allowing formation of adduct with DMF to go to completion and then cooling to a temperature preferably of about 0° C., resulting in a reaction mixture containing the said adduct (E) chlorinating sucrose-6-acetate in a solution as a preferred partially protected sugar, by adding the said solution slowly over several hours to the said reaction mixture of step D, controlling the temperature preferably to below 5° C., thereafter allowing temperature preferably to attain room temperature of about 30° C. and stirring for a period of time preferably of about 60 minutes, heating to a higher temperature preferably of about 100° C. and maintaining at that temperature for a period of time preferably of about 6 hours, further heating to a higher temperature preferably of about 114° C. and maintaining at that temperature for about 1.5 hrs, resulting in a reaction mixture containing chlorinated TGS-6-acetate, (F) cooling the said reaction mixture containing chlorinated TGS-6-acetate to a lower temperature preferably of about 60° C., neutralizing to pH of about 7 by adding about 7% ammonia solution as preferred alkali, preferably filtering to remove suspended solids (G) recovering TGS by a preferred process comprising isolating and simultaneously deacylating TGS-6-acetate to TGS by passing the filtrate through an affinity chromatographic column containing an adsorbent capable of selectively adsorbing TGS-6-acetate comprising a resin synthesized preferably from crosslinked polystyrene, having no ionic functional group such as ADS 600 resin sourced from Thermax as a preferred resin, adsorbing TGS-6-acetate on to the resin and passing off the DMF along with inorganic salts out of the resin by eluting and washing the column preferably by water, desorbing and deacylating simultaneously TGS-6-acetate using preferably about 10% ammonia solution in methanol to wash the column, neutralizing the TGS solution in ammonia methanol by addition of dilute HCl, distilling off methanol to obtain a syrup and preferably isolating TGS in a solid form by treating the said syrup with ethyl acetate and methanol as preferred solvent combination to obtain TGS powder.

4. A process of claim 3 of production of TGS-6-actetate as a preferred TGS-6-ester comprising isolating and purifying the said TGS-6-acetate from a reaction mixture of step (F) of claim 3 by using one or more of a process of isolation and purification preferably comprising steps of (i) passing the filtrate through an affinity chromatographic column containing an adsorbent capable of selectively adsorbing TGS-6-acetate comprising a resin synthesized preferably from crosslinked polystyrene, having no ionic functional group such as ADS600 resin as preferred adsorbent that is sourced from Thermax, (ii) adsorbing TGS-6-acetate on to the resin and passing off the DMF along with inorganic salts out of the resin by eluting and washing the column preferably by water, (iii) desorbing TGS-6-acetate by using an eluant capable of desorbing and eluting out TGS-6-acetate, including aqueous methanol, to wash the column, (iv) concentrating the eluted wash, and (v) isolating TGS-6-acetate as a solid by a method of recovering the same from the solvent, including process of solvent crystallization.

5. A process of claim 3 of production of a Vilsmeier reagent comprising use of thionyl chloride as a preferred sulphur containing acid chloride, the said process further comprising following sequential steps: (A) taking DMF as a preferred tertiary amide in a reactor, optionally sparging nitrogen as a preferred inert gas through the reaction mixture, (B) adding to it charcoal powder as a preferred powder for controlling gaseous emissions, (C) adding to the reaction mixture thionyl chloride preferably slowly over a period of time maintaining temperature preferably between about 35° to 40° C., (D) stirring the reaction mass for a further period of time sufficient for complete formation of chlorosulphite reagent, preferably for about 5 hours, (E) slowly increasing the temperature preferably up to 70° C. over a period of time preferably of 5 hours until sulphur dioxide was completely eliminated, (F) cooling mass to a preferred temperature of about 15° C. to allow precipitation of Vilsmeier reagent as a solid, (G) collecting the precipitate preferably by filtration under preferably an inert atmosphere further preferably under nitrogen.

6. A process of claim 3 of production of a Vilsmeier reagent comprising use of sulphuryl chloride as a preferred sulphur containing acid chloride, the said process further comprising following sequential steps: (A) taking DMF as a preferred tertiary amide in a reactor, optionally sparging nitrogen as a preferred inert gas through the reaction mixture, (B) adding to it charcoal powder as a preferred powder for controlling gaseous emissions, (C) adding to the reaction mixture sulphuryl chloride preferably slowly over a period of time maintaining temperature preferably between about 35° to 40° C., (D) stirring the reaction mass for a further period of time sufficient for complete formation of chlorosulphate reagent, preferably for about 5 hours, (E) followed either by isolating the solids of chlorosulphate reagent formed at room temperature for subsequent use as chlorination reagent or slowly increasing the temperature preferably up to 85° C. over a period of time preferably of 9 hours until sulphur trioxide was completely eliminated, (F) cooling mass to a preferred temperature of about 15° C. to allow precipitation of Vilsmeier reagent as a solid, awaiting completion of precipitation for a period of time preferably of about 3 hours, (G) collecting the precipitate preferably by filtration under preferably an inert atmosphere further preferably under nitrogen.

7. A process of claim 2, comprising adding chlorinating reagent to a solution of partially protected sugar, of production of TGS comprising use of either thionyl chloride or sulphuryl chloride as a preferred sulphur containing acid chloride to generate a chlorinating reagent, the said process further comprising following sequential steps: (A) taking DMF as a preferred tertiary amide in a reactor, optionally sparging nitrogen as a preferred inert gas through the reaction mixture, (B) adding to it charcoal powder as a preferred powder for controlling gaseous emissions, (C) adding to the reaction mixture, a solution of thionyl chloride or sulphuryl chloride as a preferred sulphur containing acid chloride slowly over a period of time maintaining temperature preferably between about 35° to 40° C., (D) maintaining the reaction mass preferably stirred for a preferred period of about 60 minutes for allowing formation of an adduct with DMF to go to completion and then cooling to a temperature preferably of about 0° C., resulting in a reaction mixture containing the said adduct (E) chlorinating sucrose-6-acetate in DMF solution as a preferred partially protected sugar, by adding the said solution slowly over several hours to the said reaction mixture of step D, controlling the temperature preferably to below 5° C., thereafter allowing temperature preferably to attain room temperature of about 30° C. and stirring for a period of time preferably of about 60 minutes, heating to a higher temperature preferably of about 100° C. and maintaining at that temperature for a period of time preferably of about 6 hours, further heating to a higher temperature preferably of about 114° C. and maintaining at that temperature for about 1.5 hrs, resulting in a reaction mixture, containing chlorinated TGS-6-acetate, (F) cooling the said reaction mixture containing chlorinated TGS-6-acetate to a lower temperature preferably of about 60° C., neutralizing to pH of about 7 by adding about 7% ammonia solution as preferred alkali, preferably filtering to remove suspended solids (g) recovering TGS by a preferred process comprising isolating and simultaneously deacylating TGS-6-acetate to TGS by passing the filtrate through an affinity chromatographic column containing an adsorbent capable of selectively adsorbing TGS-6-acetate comprising a resin synthesized preferably from crosslinked polystyrene, having no ionic functional group such as ADS 600 resin sourced from Thermax as a preferred resin, adsorbing TGS-6-acetate on to the resin and passing off the DMF along with inorganic salts out of the resin by eluting and washing the column preferably by water, desorbing and deacylating simultaneously TGS-6-acetate using preferably about 10% ammonia solution in methanol to wash the column, neutralizing the TGS solution in ammonia methanol by addition of dilute HCl, distilling off methanol to obtain a syrup and preferably isolating TGS in a solid form by treating the said syrup with ethyl acetate and methanol as preferred solvent combination to obtain TGS powder.

8. A process of chlorination of a partially protected sugar with an isolated N,N-dimethyl formiminium chloride chlorosulphate reagent reagent in one or more of an organic solvent other than Dimethylformamide (DMF) and the said organic solvent being capable of providing a liquid medium for the reaction.

9. A process of claim 8 in which:
a. the said partially protected sugar comprises a pentaester of sucrose or a 6-protected sucrose, the 6-protected sucrose further comprising a sucrose-6-ester or a sucrose-6-ether or a sucrose-6-diester, further including sucrose-6-acetate, sucrose-6-benzoate, sucrose-6,4′ diester, 2,3,6,3′,4′-pentaester, a sucrose-6-glutarate, sucrose-6-propionate, sucrose-6-laurate, sucrose-6-phthalate, a sucrose-6-methyl ether, sucrose-6-ethyl ether.
b. the said organic solvent being capable of providing a liquid medium for the reaction comprises one or more of a dimethyl sulfoxide, perchloroethylene, Toluene, Pyridine, xylene.

10. A process of claim 9 wherein: (A) isolated solid N,N-dimethyl formiminium chloride salt as a preferred Vilsmeir reagent is taken preferably in a glass lined reactor, perchloroethylene cooled to 0° C. is added as a preferred organic solvent in the reaction mass maintaining the temperature to 0° C. and preferably kept stirring, optionally sparging nitrogen as a preferred inert gas throughout the reaction, (B) adding sucrose-6-acetate as a preferred protected sugar dissolved in perchloroethylne as a preferred organic solvent and added over a period of time preferably of 10-15 hours to the reaction mass controlling the temperature preferably to below about 5° C. (C) allowing the reaction mass to attain room temperature of about 30° C. and then stirring for a period preferably for 60 minutes, (D) heating the reaction mass preferably to about 85° C. slowly over a period of time preferably of about 3 hours and maintaining for a period of time preferably for 60 minutes, (E) heating further to a preferred temperature 100° C. and maintaining for 6 hours, (F) heating further preferably to about 114° C. and maintaining for a preferred period of 1.5 hrs and cooling to a preferred temperature of 60° C., (G) neutralizing the mass by 7% ammonia solution as a preferred alkali to a preferred pH of 7.0.

11. A process of controlling prolific emission of a gaseous by-product in a reaction that is carried out at a temperature above 5 degrees to about 50 degrees C., preferably between 35-50 degrees C., by reverse addition of the tertiary amide to a solution of sulphur containing inorganic acid chloride further comprising a reaction a of a sulphur containing inorganic acid chloride with a tertiary amide with sparging of nitrogen as a preferred inert gas, resulting in a first reaction mixture containing an adduct formed between the said acid chloride and the tertiary amide, the said reaction comprising one or more of a reaction including (A) reaction of a sulphur containing inorganic acid chloride with a tertiary amide resulting in a first reaction mixture containing an adduct formed between the said acid chloride and the tertiary amide, or (B) a chlorination reaction by reacting the said adduct either after its isolation from the said first reaction mixture, the said chlorination reaction being carried in a medium suitable for progress of a chlorination reaction with a sugar or a partially protected sugar, or (C) a chlorination reaction comprising reacting the adduct existing as an in-situ reagent in the said first reaction mixture with a sugar or a partially protected sugar to prepare a chlorinated sugar derivative, or (D) heating the said first reaction mixture to form a second reaction mixture comprising decomposition of the said adduct in-situ to form a Vilsmeier reagent of general formula [XCIC═N.sup.+R.sub.2]Cl.sup.—wherein R represents an alkyl group, typically a methyl or ethyl group, and X represents a hydrogen atom or a methyl group, and optionally isolating the solid Vilsmeier reagent formed or (E) a chlorination reaction by reacting the said second reaction mixture with in-situ formed Vilsmeier reagent with a partially protected sugar to prepare a chlorinated sugar derivative, or (F) a chlorination reaction by reacting a sugar or partially protected sugar dissolved in DMF with a sulphur containing inorganic acid chloride followed optionally by isolating the said chlorinated sugar derivative or deacylating the said chlorinated sugar derivative to form Trichlorogalactosucrose (TGS).

12. A process of claim 11, wherein:
k. the said gaseous by product is an oxide of sulphur, including sulphur dioxide or sulphur trioxide,
l. the said sulphur containing inorganic acid chloride comprises one or more of a thionyl chloride, sulphuryl chloride and the like; or also shall include at least one equivalent sulphur containing halide including thionyl bromide,
m. the said tertiary amide comprising one or more of a dimethylformamide (DMF), Dimethyl acetamide, and the like,
n. the said adduct comprising N,N-dimethylformiminium chloride chlorosulphite (chlorosulphite reagent) when the acid chloride used in its generation is thionyl chloride or N,N-dimethylformiminium chloride chlorosulphate (chlorosulphate reagent) when the acid chloride used in its generation is sulphuryl chloride,
o. the said medium suitable for progress of a chlorination reaction being an organic solvent comprising one or more of a DMF, dimethyl sulfoxide, perchloroethylene, Toluene, Pyridine, xylene and the like,
p. the said sugar comprises raffinose,
q. the said partially protected sugar comprises a pentaester of sucrose or a 6-protected sucrose, the 6-protected sucrose further comprising a sucrose-6-ester or a sucrose-6-ether or a sucrose-6-diester, further including sucrose-6-acetate, sucrose-6-benzoate, sucrose-6,4′ diester, 2,3,6,3′,4′-pentaester, a sucrose-6-glutarate, sucrose-6-propionate, sucrose-6-laurate, sucrose-6-phthalate, a sucrose-6-methyl ether, sucrose-6-ethyl ether and the like,
r. the said process of purification and isolation includes one or more of a process comprising solvent extraction, column chromatography, reverse osmosis, crystallization and the like.

13. A process of claim no. 11 wherein thionyl chloride is taken as the preferred sulphur containing acid chloride, the said process further comprising following sequential steps: (A) DMF is added dropwise to the reaction mass maintaining the temperature preferably at about 35-40° accompanied by stirring for over 3 hours preferably using active cooling to avoid temperature exceeding 50° C., optionally sparging nitrogen as a preferred inert gas throughout the DMF addition, (B) the reaction mass is held at preferably at 45-50° C. for a preferred period of about 3 hours to facilitate complete removal of sulphur dioxide, (C) the reaction mass is cooled to about 0-5° C. and solution of sucrose-6-acetate as the preferred protected sugar dissolved in DMF is added drop wise under stirring maintaining the temperature preferably below 5 degrees C., (D) the reaction mass is allowed to attain room temperature and maintained for a preferred period of about 60 minutes, (E) the mass is heated to preferably to about 85° C. slowly over a preferred period of about 3 hours and maintained at that temperature for about 60 minutes, then heated to a preferred temperature of about 100° C. and maintained for a preferred period of about 6 hours, then further heated preferably to about 114° C. and maintained for a preferred period of about 1.5 hrs and cooled to a preferred temperature of 60° C., (F) neutralizing by an alkali, preferably with 7% ammonia solution, to a preferred pH of about 7.0 to get TGS-6-acetate, (G) deacylating to get TGS.

14. A process of claim 11 of production of TGS-6-actetate as a preferred TGS-6-ester comprising isolating and purifying the said TGS-6-acetate from a reaction mixture of step (F) of claim 3 by using one or more of a process of isolation and purification preferably comprising steps of (i) passing the filtrate through an affinity chromatographic column containing an adsorbent capable of selectively adsorbing TGS-6-acetate, including an adsorbent capable of selectively adsorbing TGS-6-acetate comprising a resin synthesized preferably from crosslinked polystyrene, having no ionic functional group such as ADS600 resin as preferred adsorbent that is sourced from Thermax, (ii) adsorbing TGS-6-acetate on to the resin and passing off the DMF along with inorganic salts out of the resin by eluting and washing the column preferably by water, (iii) desorbing TGS-6-acetate by using an eluant capable of desorbing and eluting out TGS-6-acetate, including aqueous methanol, to wash the column, (iv) concentrating the eluted wash, and (v) isolating TGS-6-acetate as a solid by a method of recovering the same from the solvent, including process of solvent crystallization.

15. A process of claim 11 of production of a Vilsmeier reagent from the same comprising use of thionyl chloride as a preferred sulphur containing acid chloride, the said process further comprising following sequential steps: (A) taking thionyl chloride in a reactor provided with sparging nitrogen as a preferred inert gas through the reaction mixture, (B) adding to it Dimethylformamide slowly over a period of time maintaining temperature preferably between about 35° to 40° C., (D) stirring the reaction mass for a further period of time sufficient for complete formation of chlorosulphite reagent, preferably for about 5 hours, (E) slowly increasing the temperature preferably up to 70° C. over a period of time preferably of 5 hours until sulphur dioxide was completely eliminated, (F) cooling mass to a preferred temperature of about 15° C. to allow precipitation of Vilsmeier reagent as a solid, (G) collecting the precipitate preferably by filtration under preferably an inert atmosphere further preferably under nitrogen.

16. A process of claim 11 of production of a Vilsmeier reagent comprising use of sulphuryl chloride as a preferred sulphur containing acid chloride, the said process further comprising following sequential steps: (A) taking sulphuryl chloride in a reactor, optionally sparging nitrogen as a preferred inert gas through the reaction mixture, (B) adding to the reaction mixture dimethylformamide slowly over a period of time maintaining temperature preferably between about 35° to 40° C., (D) stirring the reaction mass for a further period of time sufficient for complete formation of chlorosulphate reagent, preferably for about 5 hours, (E) followed either by isolating the solids of chlorosulphate reagent formed at room temperature for subsequent use as chlorination reagent or by slowly increasing the temperature preferably up to 85° C. over a period of time preferably of 9 hours until sulphur trioxide was completely eliminated, (F) cooling mass to a preferred temperature of about 15° C. to, allow precipitation of Vilsmeier reagent as a solid, awaiting completion of precipitation for a period of time preferably of about 3 hours, (G) collecting the precipitate preferably by filtration under preferably an inert atmosphere further preferably under nitrogen.

17. A process of claim 11 of production of TGS comprising use of either thionyl chloride or sulphuryl chloride as a preferred sulphur containing acid chloride to generate a chlorinating reagent, the said process further comprising following sequential steps: (A) taking thionyl chloride or sulphuryl chloride in a reactor, (B) optionally sparging nitrogen as a preferred inert gas through the reaction mixture, (C) adding to dimethyl formamide as a preferred tertiary amide slowly over a period of time maintaining temperature preferably between about 35° to 40° C., (D) maintaining the reaction mass preferably stirred for a preferred period of about 60 minutes for allowing formation of an adduct with DMF to go to completion and then cooling to a temperature preferably of about 0° C., resulting in a reaction mixture containing the said adduct (E) chlorinating sucrose-6-acetate in a solution as a preferred partially protected sugar, by adding the said solution slowly over several hours to the said reaction mixture of step D, controlling the temperature preferably to below 5° C., thereafter allowing temperature preferably to attain room temperature of about 30° C. and stirring for a period of time preferably of about 60 minutes, heating to a higher temperature preferably of about 100° C. and maintaining at that temperature for a period of time preferably of about 6 hours, further heating to a higher temperature preferably of about 114° C. and maintaining at that temperature for about 1.5 hrs, resulting in a reaction mixture containing chlorinated TGS-6-acetate, (F) cooling the said reaction mixture containing chlorinated TGS-6-acetate to a lower temperature preferably of about 60° C., neutralizing to pH of about 7 by adding about 7% ammonia solution as preferred alkali, preferably filtering to remove suspended solids (h) recovering TGS by a preferred process comprising isolating and simultaneously deacylating TGS-6-acetate to TGS by passing the filtrate through an affinity chromatographic column containing an adsorbent capable of selectively adsorbing TGS-6-acetate comprising a resin synthesized preferably from crosslinked polystyrene, having no ionic functional group such as ADS 600 resin sourced from Thermax as a preferred resin, adsorbing TGS-6-acetate on to the resin and passing off the DMF along with inorganic salts out of the resin by eluting and washing the column preferably by water, desorbing and deacylating simultaneously TGS-6-acetate using preferably about 10% ammonia solution in methanol to wash the column, neutralizing the TGS solution in ammonia methanol by addition of dilute HCl, distilling off methanol to obtain a syrup and preferably isolating TGS in a solid form by treating the said syrup with ethyl acetate and methanol as preferred solvent combination to obtain TGS powder.
